(19) United States
(12) Patent Application Publication
(10) Pub. No.: US 2009/0223823 A1
(43) Pub. Date: Sep. 10, 2009

(54) METHOD OF SEPARATING PROTEIN, METHOD OF STAINING PROTEIN AND LIQUID PROTEIN-STAINING AGENT AND PROTEIN-STAINING KIT TO BE USED IN THESE METHODS

(75) Inventors: Ichiji Namatame, Tokyo (JP); Yoshinori Ishii, Tokyo (JP); Yoshimasa Saito, Tokyo (JP); Takayoshi Komatsu, Ibaraki (JP); Yasuhiro Ogawa, Tokyo (JP); Takashi Shibata, Tokyo (JP); Hideki Kinoshita, Ibaraki (JP); Kenji Yokoyama, Ibaraki (JP)

Correspondence Address: LEYDIG VOIT & MAYER, LTD, TWO PRUDENTIAL PLAZA, SUITE 4900, 180 NORTH STETSON AVENUE, CHICAGO, IL, 60601-6731, US

(73) Assignees: National Institute of Advanced Industrial Science and Technology, Tokyo (JP); Sharp Corporation, Osaka-shi (JP); Toppan Printing Co., Ltd., Tokyo (JP); Katayanagi Institute, Hachioji-shi (JP)

(21) Appl. No.: 11/720,927

(22) PCT Filed: Dec. 6, 2005
(86) PCT No.: PCT/JP2005/022724

§ 371 Date: Oct. 10, 2007

(30) Foreign Application Priority Data
Dec. 6, 2004 (JP) 2004-353395

(51) Int. Cl. B01D 57/02; C07K 2/00; G01N 33/00

(52) U.S. Cl. 204/469; 530/300; 252/408.1

(57) ABSTRACT

A method of separating a protein by gel electrophoresis, which comprises a staining step for bringing a protein-containing sample into contact with a staining liquid containing a staining agent, a surfactant and a buffer solution, and an electrophoresis step for subjecting the protein-containing sample after the staining step to gel electrophoresis.

According to the present invention, using a staining liquid containing a surfactant such as SDS and the like, a protein-containing sample can be stained in a short time, and can develop color with high sensitivity. In addition, since an excess staining agent migrates earlier than protein by electrophoresis, a washing operation is not necessary. Furthermore, by staining, after the first dimension electrophoresis, with a staining liquid containing a surfactant such as SDS and the like, the second dimension electrophoresis can be performed immediately. As a result, the number of steps can be reduced as compared to conventional protein separation methods, and the separation operation can be simplified. Hence, a method capable of staining and separating a protein conveniently and quickly by electrophoresis can be provided.

### TECHNICAL FIELD

The present invention relates to a separation method and a staining method of a protein in electrophoresis, and a protein-staining liquid and a protein-staining kit to be used for these methods.

### BACKGROUND ART

As a method for best separating each component in a mixture containing a protein, electrophoresis is known. Of electrophoreses, two-dimensional gel electrophoresis is widely used since it can separate a cell crude extract into as many as 1,000 respective protein components. The two-dimensional gel electrophoresis includes, for example, treating a protein-containing sample after first dimension electrophoresis with sodium dodecylsulfate (SDS), subjecting the sample to second dimension electrophoresis, staining and washing the separated protein, and analyzing the protein. For staining a protein, the CBB staining method, the Sypro Ruby staining method and the like are used (Sugano, “Electrophoresis New Protocol (Denkieidou Saishin Purotokoru)”, Youdosha, Jan. 1, 2000).

However, since many of the electrophoreses described in the above-mentioned literature require one to several hours for a staining operation and indispensably require a washing operation after staining, there is a problem of the staining operation requiring too long time.

In recent years, to solve such problems, an Ettan DIGE method capable of performing efficient staining has been proposed (Novel experimental design for comparative two-dimensional gel analysis: two-dimensional difference gel electrophoresis incorporating a pooled internal standard. Proteomics. 3, 36-44 (2003)). Ettan DIGE method is a method including reacting a protein-containing sample with a staining agent, quenching the reaction, subjecting the reaction mixture to the first dimension electrophoresis, subjecting the protein-containing sample after electrophoresis to an SDS treatment, and subjecting the sample to the second dimension electrophoresis to analyze the protein.

### DISCLOSURE OF THE INVENTION

While the Ettan DIGE method can perform the staining operation in a short period of time as compared to conventionally-known electrophoretic analyses, it requires quenching the reaction between an excess staining agent and the protein to control the amount of the staining agent. This renders the staining operation complicated. As mentioned above, since the staining operation in conventional electrophoretic analyses is complicated, there is a demand for a method capable of conveniently and quickly performing staining and separation of a protein.

The present invention has been made in view of such actual situation, and its problem to be solved is provision of a method capable of conveniently and quickly performing staining and separation of a protein in electrophoresis, and a protein-staining liquid and a protein-staining kit to be used for the method.

The present inventors have conducted intensive studies in an attempt to solve the above-mentioned problem and found that a staining time can be shortened and the staining sensitivity can be improved by staining using a staining liquid containing a staining agent, a surfactant and a buffer solution, which resulted in the completion of the present invention.

Accordingly, the present invention is characterized by the following.

(1) A method of separating a protein by gel electrophoresis, which comprises a staining step for bringing a protein-containing sample into contact with a staining liquid containing a staining agent, a surfactant and a buffer solution, and an electrophoresis step for subjecting the protein-containing sample after the above-mentioned staining step to gel electrophoresis.
(2) The method of the above-mentioned (1), wherein the above-mentioned surfactant is a dodecylsulfate alkali metal salt.
(3) The method of the above-mentioned (2), wherein the above-mentioned dodecylsulfate alkali metal salt is sodium dodecylsulfate or lithium dodecylsulfate.
(4) The method of the above-mentioned (2) or (3), wherein the concentration of the above-mentioned dodecylsulfate alkali metal salt in the above-mentioned staining liquid is 0.5-10%.
(5) The method of any one of the above-mentioned (1)-(4), wherein the concentration of the above-mentioned buffer solution in the above-mentioned staining liquid is not more than 10 mM.
(6) The method of any one of the above-mentioned (1)-(5), wherein the above-mentioned staining agent is a covalently-bonding staining agent.
(7) The method of the above-mentioned (6), wherein the above-mentioned covalently-bonding staining agent is an amino group-modifying staining agent.
(8) The method of the above-mentioned (7), wherein the above-mentioned amino group-modifying staining agent is a cyanine pigment.
(9) The method of any one of the above-mentioned (1)-(8), wherein the above-mentioned gel electrophoresis is SDS-polyacrylamide gel electrophoresis.
(10) The method of any one of the above-mentioned (1)-(9), which further comprises a step for subjecting the protein-containing sample to electrophoresis before the above-mentioned staining step, wherein the protein-containing sample in the above-mentioned staining step is the protein-containing sample after the above-mentioned electrophoresis.
(11) The method of the above-mentioned (10), wherein the above-mentioned electrophoresis is isoelectric focusing electrophoresis.
(12) A method of staining a protein in gel electrophoresis, which comprises bringing a protein-containing sample into contact with a staining liquid containing a staining agent, a surfactant and a buffer solution before gel electrophoresis.
(13) The method of the above-mentioned (12), wherein the above-mentioned electrophoresis is two-dimensional electrophoresis comprising the first dimension electrophoresis and the second dimension electrophoresis, and the protein-containing sample after the completion of the above-mentioned first dimension electrophoresis is brought into contact with the above-mentioned staining liquid before the above-mentioned second dimension electrophoresis.
(14) A protein-staining kit for gel electrophoresis, which comprises a buffer solution, a staining agent and a surfactant.
(15) The protein-staining kit of the above-mentioned (14), further comprising alcohol.
(16) The protein-staining kit of the above-mentioned (14) or (15), further comprising a written document containing an explanation of the separation method of any one of the above-mentioned (1)-(11), or the staining method of the above-mentioned (12) or (13).
(17) A protein-staining liquid for gel electrophoresis, comprising a staining agent, a surfactant and a buffer solution.
(18) The protein-staining liquid of the above-mentioned (17), further comprising alcohol.

### BRIEF DESCRIPTION OF THE DRAWINGS

FIG. 1 shows an image of two-dimensional electrophoresis of the mouse brain tissue extract stained in Example 1 by the staining method of the present invention.

FIG. 2 shows an image of two-dimensional electrophoresis of the mouse brain tissue extract stained in Example 2 by the staining method of the present invention.

FIG. 3 shows an image of SDS-PAGE electrophoresis of the mouse brain tissue extract stained in Example 3 by the staining method of the present invention.

FIG. 4 shows an image of SDS-PAGE electrophoresis of the mouse brain tissue extract stained in Example 4 by the staining method of the present invention.

FIG. 5 shows an image of two-dimensional electrophoresis of the mouse brain tissue extract stained in Comparative Example 1 with a staining liquid free of SDS.

FIG. 6 shows an image of two-dimensional electrophoresis of the mouse brain tissue extract stained in Comparative Example 2 by the CBB staining method.

FIG. 7 shows images of two-dimensional electrophoresis of the mouse brain tissue extracts stained in Comparative Example 3 by the staining method of the present invention and the Ettan DIGE method.

### BEST MODE FOR EMBODYING THE INVENTION

The present invention is explained in detail in the following by referring to preferable embodiments.

The method of separating a protein by gel electrophoresis according to the present invention (hereinafter to be simply referred to as “the separation method”) characteristically includes a staining step and an electrophoresis step. The staining step includes contacting a protein-containing sample with a staining liquid containing a staining agent, a surfactant and a buffer solution. The electrophoresis step includes subjecting the protein-containing sample after the staining step to gel electrophoresis.

The staining step is explained first. In the staining step, a protein-containing sample is contacted with a staining liquid before applying the protein-containing sample to a support. As the protein-containing sample, an extract of a biological sample containing plural kinds of proteins can be used. As the biological sample, for example, biologic cell of human, domestic animals and poultry such as bovine, horse, swine, sheep, dog, bird and the like, experiment animals such as mouse, rat and the like, and the like, tissues containing them (e.g., hepatic tissue, muscular tissue, brain tissue, cardiac tissue, blood, plasma, serum, body fluid such as lymph fluid and the like, lymph node) or secretion (e.g., urine) and the like can be mentioned. When the spot or band of a protein after gel electrophoresis exceeds detection limits, it is desirable to subject the protein-containing sample to separation and purification, a fractionation treatment and the like in advance.

As the support, gels such as polyacrylamide gel, agarose gel and the like can be used. While the gel concentration of the support can be appropriately selected according to the molecular weight of the protein to be separated, for example, it is generally 3-20% in the case of a polyacrylamide gel. When the molecular weight of the protein to be separated is unknown or the molecular weight varies over a wide range, for example, a gel having a density gradient of 5-20% may be used. As the aforementioned gel, a gel having a desired concentration may be prepared, for example, by polymerization of acrylamide and N,N-methylenebisacrylamide. Alternatively, a commercially available precast gel such as SureBlot gel (manufactured by Fujisawa Pharmaceutical Co., Ltd.), Ready Gel (manufactured by BIO-RAD), Immobiline Dry strip gel, Ettan DALT gel, Multiphor II Precast gel, Phasts system Precast gel and Genephor Precast gel (all of which are manufactured by Amersham Biosciences), NuPAGE Bis-Tris gel, NuPAGE Tris-Acetate gel, Tris-Glycine gel, Tricine gel, IEF gel, E-GEL gel, TBE gel and TBE-Urea gel (all of which are manufactured by Invitrogen), PAG mini gel (manufactured by Daiichi Pure Chemicals Co., Ltd.), PAGEL and e-PAGEL (both of which are manufactured by ATTO CORPORATION), XV PANTERA gel and Perfect NT gel (both of which are manufactured by DRC) and the like may be obtained. In the present specification, the gel concentration (%) of support means w/v %.

As the staining liquid for staining a protein-containing sample, the protein-staining liquid for gel electrophoresis in the present invention (hereinafter to be simply referred to as “the staining liquid”) can be used. The staining liquid of the present invention characteristically contains a staining agent, a surfactant and a buffer solution.

As the buffer solution, various buffer solutions known in the pertinent technical field can be used. A buffer solution that does not inhibit the reaction of a functional group of the staining liquid with the functional group of a protein reactive with the above-mentioned functional group is preferable. Such buffer solution can be appropriately selected in consideration of the type of the staining agent to be used and, for example, carbonate buffer solution (containing Na₂CO₃ and NaHCO₃ in combination), phosphate buffer solution (containing Na₂HPO₄ and NaH₂PO₄ in combination), Clark and Lubs solution (containing KH₂PO₄ and NaOH in combination), NaHCO₃ buffer solution (wherein pH is controlled with 5% CO₂ or NaOH), imidazole-HCl buffer solution (2,4,6-trimethylpyridine-HCl buffer solution), morpholinopropanesulphonic acid (MOPS)-KOH buffer solution, barbital-HCl buffer solution (containing sodium 5,5-diethylbarbiturate and HCl in combination), N-ethylmorpholine-HCl buffer solution, N-2-hydroxyethylpiperazine-N′-ethanesulfonic acid (HEPES)-NaOH buffer solution, N-2-hydroxyethylpiperazine-N′-3-propanesulfonic acid (EPPS)-NaOH buffer solution, N,N-(bis-2-hydroxymethyl)glycine (BICINE)-NaOH buffer solution, Tris-glycine buffer solution, Tris-HCl buffer solution, Tris-acetate buffer solution, MES (2-morpholinoethanesulfonic acid) buffer solution and TRICINE buffer solution can be mentioned. Of these, for example, when an amino group-modifying staining agent (e.g., Cy5, Cy7) is used such as N-hydroxysuccinimide series, carbonate buffer solution and NaHCO₃ buffer solution are preferable. The concentration of the buffer solution in the staining liquid is preferably not more than 10 mM, more preferably not more than 3 mM. When the concentration exceeds 10 mM, band skewing of the protein separated by electrophoresis tends to occur. Since the band skewing may occur when the concentration of the buffer solution is too low, the concentration of the buffer solution is desirably set to not less than 0.3 mM.

As the staining agent, a covalently-bonding staining agent is preferable. Here, the covalently-bonding staining agent refers to a staining agent having a reactive group capable of forming a covalent bond by a chemical reaction with a functional group (e.g., NH₂ group, COOH group, SH group, OH group and the like) present in a structure such as an organic compound, a nucleic acid, a protein and the like, such as isothiocyanate group, STP ester group, sulfonyl chloride group, N-hydroxysuccinimidyl (NHS) ester group, alkyl halide group, maleimide group, symmetric disulfide group and the like, in the structure thereof. As the staining agent, for example, cyanine pigments (e.g., Cy5, Cy3, Cy2, Cy7 (manufactured by Amersham Bioscience)), Alexa Fluors, Biotins, BODIPYs, Fluoresceins, Oregon Greens, Rhodamines, Texas reds, Coumarins, NBDs (7-nitrobenz-2-oxa-1,3-diazole) and the like can be mentioned. Of these covalently-bonding staining agents, an amino group (NH₂)-modifying staining agent is preferable and, cyanine pigments, particularly Cy5, Cy3 and Cy2 (manufactured by Amersham Bioscience), are preferable. The concentration of the staining agent in the staining liquid is preferably 100-1,000 μg/mL, more preferably 200-500 μg/mL. When the concentration is less than 100 μg/mL, the fluorescence sensitivity tends to remarkably decrease and when it exceeds 1,000 μg/mL, the fluorescence sensitivity tends to be saturated. In addition, a non-covalently-bonding staining agent can also be used in the present invention and, for example, Sypro Orange and Sypro red (both of which are manufactured by Molecular Probes) can be used.

As the surfactant, a surfactant capable of negatively charging a protein is preferable and, for example, an anion surfactant can be mentioned. As the surfactant, for example, a dodecyl sulfate alkali metal salt can be mentioned and, specifically, sodium dodecylsulfate (SDS), lithium dodecylsulfate (LDS) and the like can be mentioned. While the surfactants such as SDS and the like also function as protein solubilizers, staining and SDS denaturation of a protein can be simultaneously performed by the co-presence of SDS and a staining agent. As a result, convenient and rapid staining of a protein-containing sample becomes possible. The concentration of the surfactant in the staining liquid is, for example, preferably 0.5-10%, more preferably 1.0-5.0%, still more preferably 1.0-2.0%, in the case of a dodecyl sulfate alkali metal salt. When the concentration is less than 0.5%, the fluorescence sensitivity tends to decrease and when it exceeds 10%, the fluorescence sensitivity tends to be saturated. In the present specification, the concentration (%) of the surfactant means w/v %.

The staining liquid of the present invention may further contain alcohol. The contained alcohol can improve the protein staining efficiency. While the factor contributing to such effect has not been clearly elucidated, the present inventors assume that improved permeability of a staining liquid into the gel is one of the factors. The alcohol is preferably a straight chain or branched alcohol having 1 to 4 carbon atoms and, specifically, methanol, ethanol, propanol, iso-propanol, butanol sec-butanol and the like can be mentioned. Of these, methanol, ethanol, propanol and butanol are preferable, and methanol, ethanol and propanol are more preferable. The concentration of alcohol in the staining liquid is preferably 0.5-30%, more preferably 0.5-10%, further preferably 0.5-1.0%. When the concentration is less than 0.5%, the effect provided by the addition of alcohol tends to become insufficient, and when it exceeds 30%, the staining efficiency tends to decrease. In the present specification, the concentration (%) of alcohol means v/v %.

The pH of the staining liquid in the present invention can be appropriately selected according to the staining agent to be used and, for example, when an amino group-modifying staining agent such as N-hydroxysuccinimide type Cy5, Cy3 and Cy2 is used, the pH of the staining liquid is preferably 9.5-10.0.

For the contact of a protein-containing sample with the staining liquid, the method of staining protein in the gel electrophoresis in the present invention (hereinafter to be simply referred to as “the staining method”) can be applied. The staining method of the present invention characteristically includes contacting a protein-containing sample with the staining liquid before gel electrophoresis, and simultaneously performing staining and a surfactant treatment (SDS denaturation etc.) of the protein. When the gel electrophoresis is two-dimensional electrophoresis, a protein-containing sample after the first dimension electrophoresis is brought into contact with a staining liquid, before the second dimension electrophoresis.

A protein-containing sample can be brought into contact with a staining liquid by a convenient means such as dropwise addition of a staining liquid using a pipette to a gel supporting a protein-containing sample, immersion of a gel in a staining liquid and the like. The contact time of the staining liquid and the protein-containing sample may be about 30 minutes. Even when electrophoresis is performed immediately after the contact, sufficient staining can be afforded. Since the staining agent and the protein have markedly different molecular weights and the rate of negative charge to the molecular weight (negative charge/molecular weight) differs strikingly between them, the electrophoretic mobility of the two differs greatly from each other. As a result, since an excess amount of the staining agent migrates by electrophoresis before the protein, detection of the protein becomes possible without an influence of the excess staining agent. In addition, a washing operation becomes unnecessary. Consequently, the protein can be stained conveniently and rapidly. In contrast, conventional staining methods require reaction of a staining agent and a protein for a sufficient time, and also require a washing operation, which in turn results in an inconvenience in that the staining operation is complicated and time-consuming. A comparison of the staining time by the staining method of the present invention and conventionally-known protein staining methods is shown in Table 1.

TABLE 1

| | | | stain- |
|---|---|---|---|
| | | steps in staining | ing |
| staining | timing of | operation | time |
| method | staining | (operation time; min) | (min) |
| present | staining before | staining alone | <30 |
| invention | electrophoresis | | |
| CCB | staining after | washing(15) → staining(60) | 105 |
| | electrophoresis | → washing(30) | |
| silver | staining after | immobilizing(20) → washing | 75 |
| staining | electrophoresis | (20) → staining(20) → | |
| | | quenching(15) | |
| Sypro | simultaneous | staining(40) → washing(15) | 55 |
| Orange | staining with | | |
| | electrophoresis | | |
| Sypro Red | simultaneous | staining(40) → swashing(15) | 55 |
| | staining with | | |
| | electrophoresis | | |
| Sypro | staining after | washing(30) → staining | 240 |
| Ruby | electrophoresis | (180) → washing(30) | |
| Ettan | staining before | reaction(30) → | 40 + α |
| DIGE | electrophoresis | quenching(10) | |

The electrophoresis step is explained below. In the electrophoresis step, a protein-containing sample subjected to the protein staining and surfactant treatment (SDS denaturation etc.) in the staining step is applied to gel electrophoresis. In the present invention, electrophoresis can be performed according to a conventionally-known method. While the gel electrophoresis is not particularly limited as long as it can separate a protein and, for example, isoelectric focusing electrophoresis, sodium dodecylsulfate (SDS) polyacrylamide gel electrophoresis (SDS-PAGE), disc gel electrophoresis, slab gel electrophoresis and gel isotachophoresis can be mentioned. Of these, SDS-PAGE and isoelectric focusing electrophoresis are preferable in the case of one-dimensional electrophoresis. In the case of two-dimensional electrophoresis, for example, two electrophoreses capable of separating a protein based on two different factors of isoelectric point and molecular weight are preferably combined. Specifically, it is preferable to select isoelectric focusing electrophoresis for the first dimension and SDS-PAGE for the second dimension.

While isoelectric focusing electrophoresis requires formation of a gel gradient in the gel, the gel gradient can be formed by a method including addition of carrier ampholytes to polyacrylamide gel to apply an electric field, a method including formation of pH gradient using acrylamide compounds having various isoelectric points, simultaneously with preparation of the gel and the like can be mentioned. In two-dimensional electrophoresis using isoelectric focusing electrophoresis and SDS-PAGE, a gel containing a protein separated by isoelectric focusing electrophoresis is placed on the end of gel on the second dimension, and allowed to migrate in the direction orthogonal to the developing direction of isoelectric focusing electrophoresis, whereby the protein is separated. The energizing conditions for gel electrophoresis can be appropriately set according to the electrophoresis and the like. For example, in the case of isoelectric focusing electrophoresis using a ZOOM IPG runner system (manufactured by Invitrogen), for example, the conditions involving stepwise raising of the voltage such as 200V 20 minutes, 450V 15 minutes, 750V 15 minutes and 2000V 30 minutes are employed. In the case of SDS-PAGE, electrophoresis is performed under 200V energizing condition.

The protein separated in the aforementioned electrophoresis step can be detected by, for example, incising the band alone with a utility knife etc., and measuring the fluorescence strength at a particular wavelength using a fluorescence spectrometer. The protein detected in this way is decolorized, where necessary, and recovered by extraction, transcription to a membrane and the like, thus permitting the analysis of the protein in terms of molecular weight, purity, identification, quantification and the like.

As an embodiment of the protein-staining kit of the present invention, a kit comprising a composition containing a staining agent, a composition containing a surfactant and, where necessary, a buffer solution and/or alcohol, in separate containers can be mentioned. By mixing them when a protein is to be stained, a staining liquid having a desired concentration and a desired pH can be preferably prepared. As another embodiment, a protein-staining kit comprising a first section for placing a composition containing a staining agent, a second section for placing a composition containing a surfactant, where necessary, a third section for placing a buffer solution and/or a fourth section for placing alcohol, in a container partitioned in multiple compartments can be mentioned. Moreover, the protein-staining kit of the present invention may further contain a written document with an explanation relating to the aforementioned separation method and staining method of the present invention. The composition containing a staining agent and the composition containing a surfactant are preferably adjusted to desired concentrations in advance.

### EXAMPLES

The present invention is explained in detail in the following by referring to Examples, which are not to be construed as limitative.

### Example 1

A solution of mouse brain tissue extract (5.2 mg protein/ml 50 mM Tris-HCl (pH 7.6)/20% glycerol/0.3 M sodium chloride/protease inhibitor cocktail 1 tab/10 mL (Roche-diagnostics)) was subjected to separation by two-dimensional electrophoresis. To be specific, the extract (7.7 μL) was added to a swelling liquid (6M Urea/2 M thiourea/2% CHAPS solution 191 μL, ampholyte (pH 3-10) 1 μL, 0.1% bromophenol blue 4 μL, 1M dithiothreitol 4 μL), and admixed. Using the solution (155 μL), immobiline pH gradient (IPG) gel strip (Invitrogen) having a pH gradient of pH 3-10 was swollen for 16 hr. After swelling, the first dimension isoelectric focusing electrophoresis (ZOOM IPG Runner System) was performed under the conditions of 200 V 20 min, 450 V 15 min, 750 V 15 min and 2000 V 30 min, while stepwisely elevating the voltage.

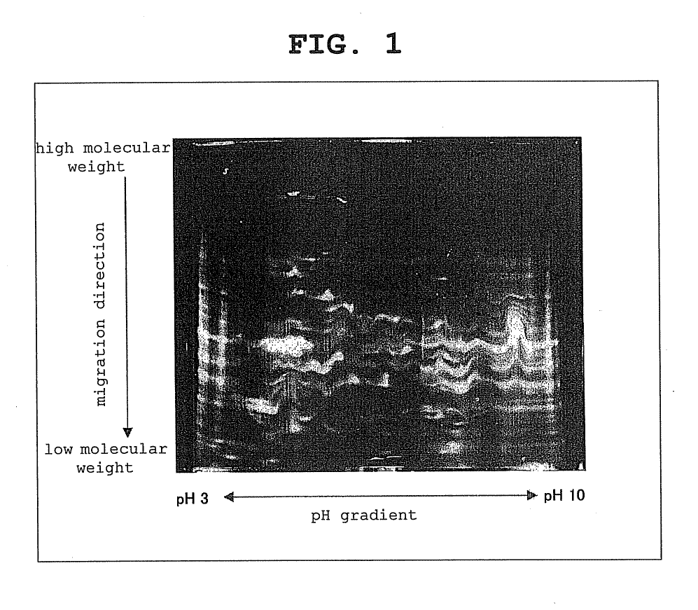

After the electrophoresis, a staining liquid using a fluorescent reagent (N-hydroxysuccinimide type Cy5 (Amersham Biosciences)), that covalently binds with the amino group of a protein under alkali conditions, (97-377 μg/mL 2% SDS/100 mM Na₂CO₃/NaHCO₃ (pH 9.9), 50 μL) was added dropwise to the IPG gel strip with a pipetman for staining, and the strip was immediately applied to the second dimension 4-12% gradient SDS-polyacrylamide electrophoresis gel, and electrophoresis was performed under the conditions of 200 V 40 min. Since free Cy5 unreactive with the protein has a low molecular weight, it migrates earlier to the anode side as a visible light blue band. After the gel electrophoresis, the main band on the anode end was cut with a utility knife etc., and the fluorescence intensity at 680 nm was measured using a fluorescence image analyzer ProExpress (Perkin-Elmer) at an excitation wavelength 625 nm, based on which the migration image of the protein was observed. As a result, when a Cy5 staining liquid containing 2% SDS was used, protein staining was confirmed to have been improved, though the band was skewed (FIG. 1). In addition, a migration pattern same as the one obtained by Coomassie Brilliant Blue (CBB) staining method wherein a gel after two-dimensional electrophoresis is processed for about 105 min was observed. The concentration (%) of glycerol is in w/v %, which is the same for the following.

### Example 2

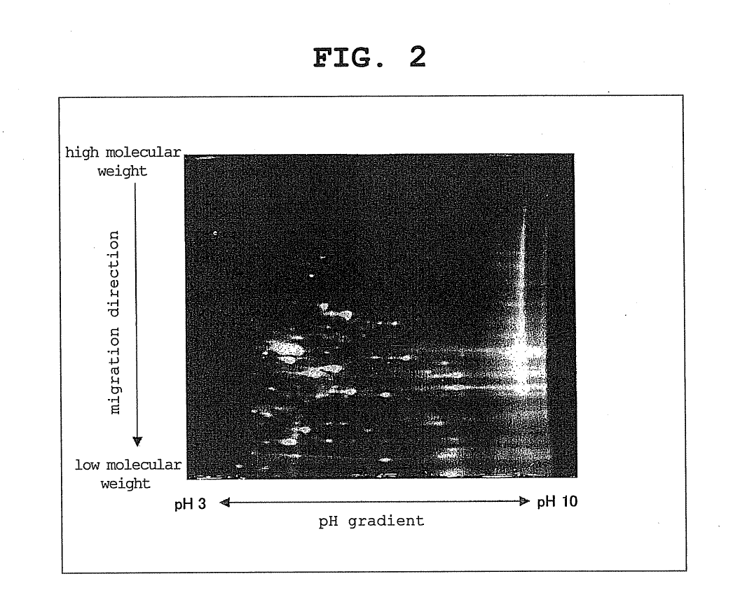

In the same manner as in Example 1 except that the sodium carbonate buffer solution was changed to 3 mM Na₂CO₃/NaHCO₃, two-dimensional electrophoresis was performed and the migration image of the protein was observed. As a result, it was confirmed that the band skewing could be eliminated while maintaining the staining efficiency of the protein (FIG. 2). As in Example 1, a migration pattern same as the one obtained by CBB staining method was observed.

### Example 3

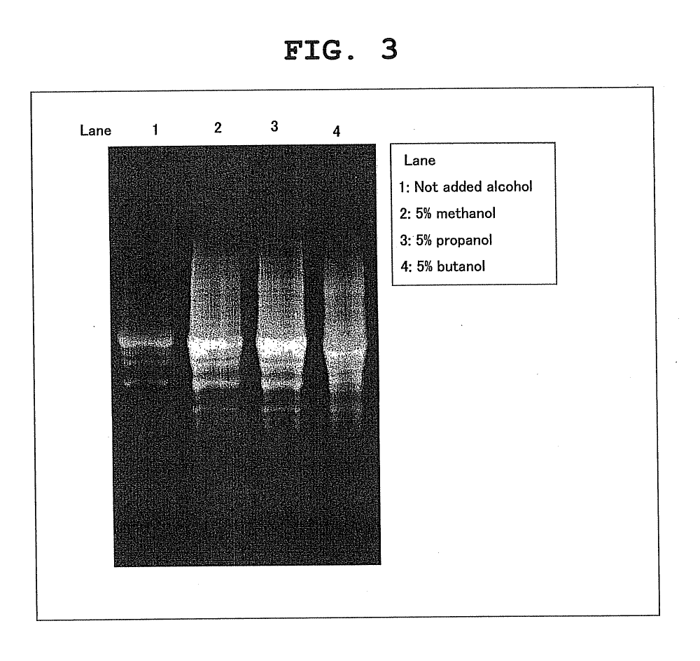

A solution of mouse brain tissue extract (5 mg protein/ml 50 mM Tris-HCl (pH 7.6)/20% glycerol/0.3 M sodium chloride/protease inhibitor cocktail 1 tab/10 mL (Roche-diagnostics)) was subjected to separation by SDS-PAGE. To be specific, to this extract (10 μL) was added a N-hydroxysuccinimide type Cy5 (manufactured by Amersham Biosciences) staining liquid (10 μL, 1 mg/mL 2% SDS/100 mM Na₂CO₃/NaHCO₃ (pH 9.9)), and the mixture was incubated at room temperature for 30 min for staining. This solution (10 μL) was applied to 10-20% gradient SDS-polyacrylamide electrophoresis gel (Sure Blot F1 gel, manufactured by Fujisawa Pharmaceutical Co., Ltd.), and electrophoresis was performed under the conditions of 240 V 15 min. After the electrophoresis, the gel was washed with 10% methanol/7% aqueous acetate solution for 1 hr, and the fluorescence intensity at 680 nm was measured using a fluorescence image analyzer ProExpress (Perkin-Elmer) at an excitation wavelength 625 nm, based on which the migration image of the protein was observed. In addition, staining and electrophoresis were performed under similar conditions and using staining liquids obtained by adding 5% each of methanol, propanol and butanol to staining liquids. The results of the extract free of alcohol are shown in lane 1 of FIG. 3, the results of the extract added with methanol are shown in lane 2 of FIG. 3, the results of the extract added with propanol are shown in lane 3 of FIG. 3, and the results of the extract added with butanol are shown in lane 4 of FIG. 3. A comparison with the extract free of alcohol has revealed that protein bands can be detected with high sensitivity when methanol, propanol and butanol are added to the extracts. The concentration (%) of acetate is in v/v %, which is the same for the following.

### Example 4

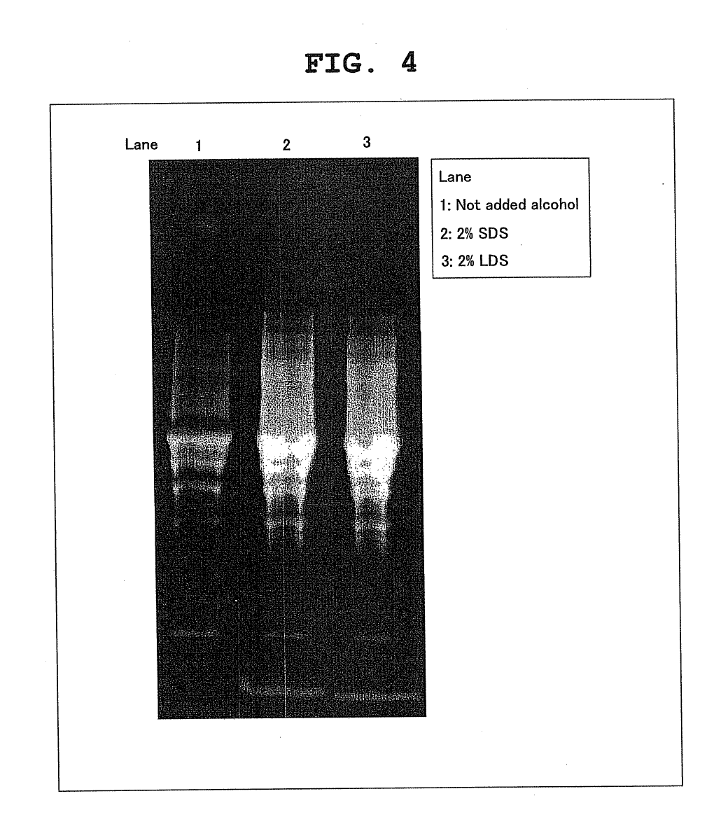

A solution of mouse brain tissue extract (5 mg protein/ml 50 mM Tris-HCl (pH 7.6)/20% glycerol/0.3 M sodium chloride/protease inhibitor cocktail 1 tab/10 mL (Roche-diagnostics)) was subjected to separation by SDS-PAGE. To be specific, to this extract (10 μL) was added a N-hydroxysuccinimide type Cy5 (manufactured by Amersham Biosciences) staining liquid (10 μL, 1 mg/mL 2% SDS/100 mM Na₂CO₃/NaHCO₃ (pH 9.9)), and the mixture was incubated at room temperature for 30 min for staining. This solution (10 μL) was applied to 10-20% gradient SDS-polyacrylamide electrophoresis gel (Sure Blot F1 gel, manufactured by Fujisawa Pharmaceutical Co., Ltd.), and electrophoresis was performed under the conditions of 240 V 15 min. After the electrophoresis, the gel was washed with 10% methanol/7% aqueous acetate solution for 1 hr, and the fluorescence intensity at 680 nm was measured using a fluorescence image analyzer ProExpress (Perkin-Elmer) at an excitation wavelength 625 nm, based on which the migration image of the protein was observed. When 2% lithium dodecylsulfate (LDS) was added to a staining liquid instead of 2% SDS, and the mixture was stained under similar conditions. The migration results were compared. As a result, it was clarified that the protein band could be detected with higher sensitivity in the case of 2% LDS addition (FIG. 4 lane 3) than 2% SDS addition (FIG. 4 lane 2).

### Comparative Example 1

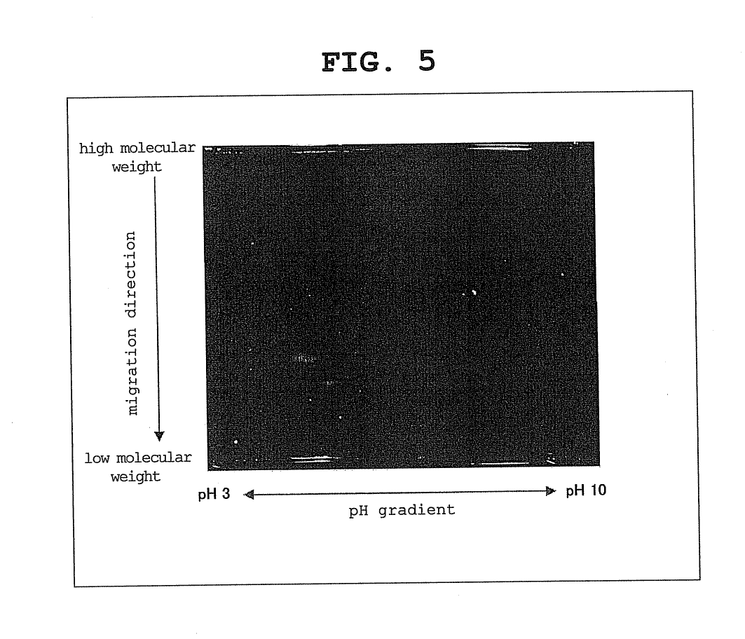

In the same manner as in Example 1 except that a staining liquid free of 2% SDS was used and the second dimension electrophoresis was performed after 30 min from the staining, two-dimensional electrophoresis was performed and the migration image of the protein was observed. As a result, when a staining liquid free of 2% SDS was used, a stained protein was hardly observed (FIG. 5).

### Comparative Example 2

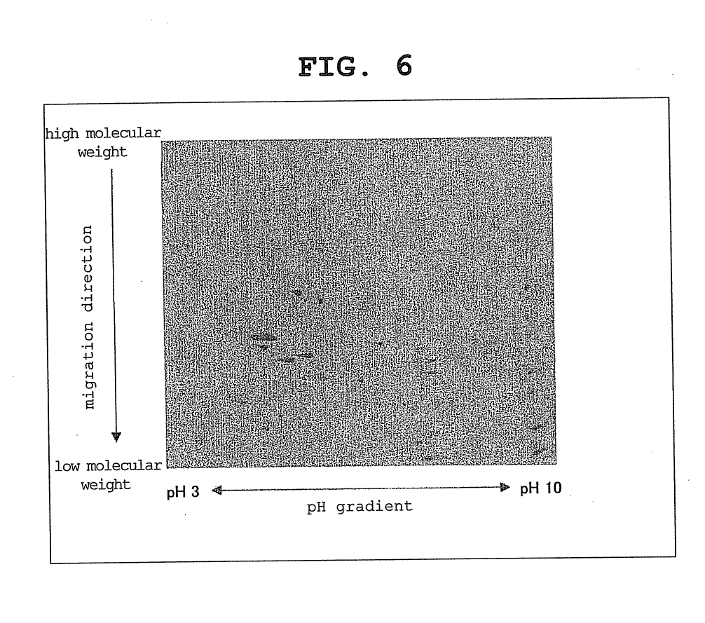

The Coomassie Brilliant Blue (CBB) staining method wherein a gel after two-dimensional electrophoresis is processed for about 105 min was performed. For CBB staining, Biosafe-CBB (manufactured by BIO-RAD) was used. That is, the gel after electrophoresis was washed three times with H₂O for 5 min each, stained with a Biosafe-CBB solution for 60 min, and washed with H₂O for 30 min to complete the staining. The migration pattern is shown in FIG. 6.

### Comparative Example 3

An extract solution of mouse liver tissue (66 mg protein/mL 50 mM Tris-HCl (pH 8.5)/20% glycerol/0.3 M sodium chloride/protease inhibitor cocktail (manufactured by Roche-diagnostics) 1 tab/10 mL) was stained by the staining method of the present invention or the Ettan DIGE method and separated by two-dimensional electrophoresis. The Ettan DIGE method was performed according to a conventional method. That is, to this extract (1.5 μL, corresponding to 100 μg protein amount) was added a 1 mM CyDye DIGE Fluor minimal dye (manufactured by Amersham Biosciences)/dimethyl formamide solution (2 μL), and the mixture was reacted for 30 min at room temperature. A 10 mM aqueous lysine solution (2 μL) was added and the mixture was reacted on ice for 10 min to quench the reaction. Thereto was added 7 M urea/2 M thiourea/4% CHAPS to increase the amount to 100 μL, and the mixture was subjected to two-dimensional electrophoresis as a Cy5 labeled protein solution. To be specific, a Cy5 labeled protein solution (20 μl) was added to a swelling liquid (6 M urea/2 M thiourea/2% CHAPS solution 191 μL, ampholyte (pH 3-10) 1 μL, 0.1% bromophenol blue 4 μL, 1 M dithiothreitol 4 μL) and mixed. Using this solution (155 μL), an immobiline pH gradient (IPG) gel strip (Invitrogen) having a pH gradient of pH 3-10 was swollen for 16 hr. After swelling, the first dimension isoelectric focusing electrophoresis (ZOOM IPG Runner System) was performed under the conditions of 200 V 20 min, 450 V 15 min, 750 V 15 min and 2000 V 30 min, while stepwisely elevating the voltage.

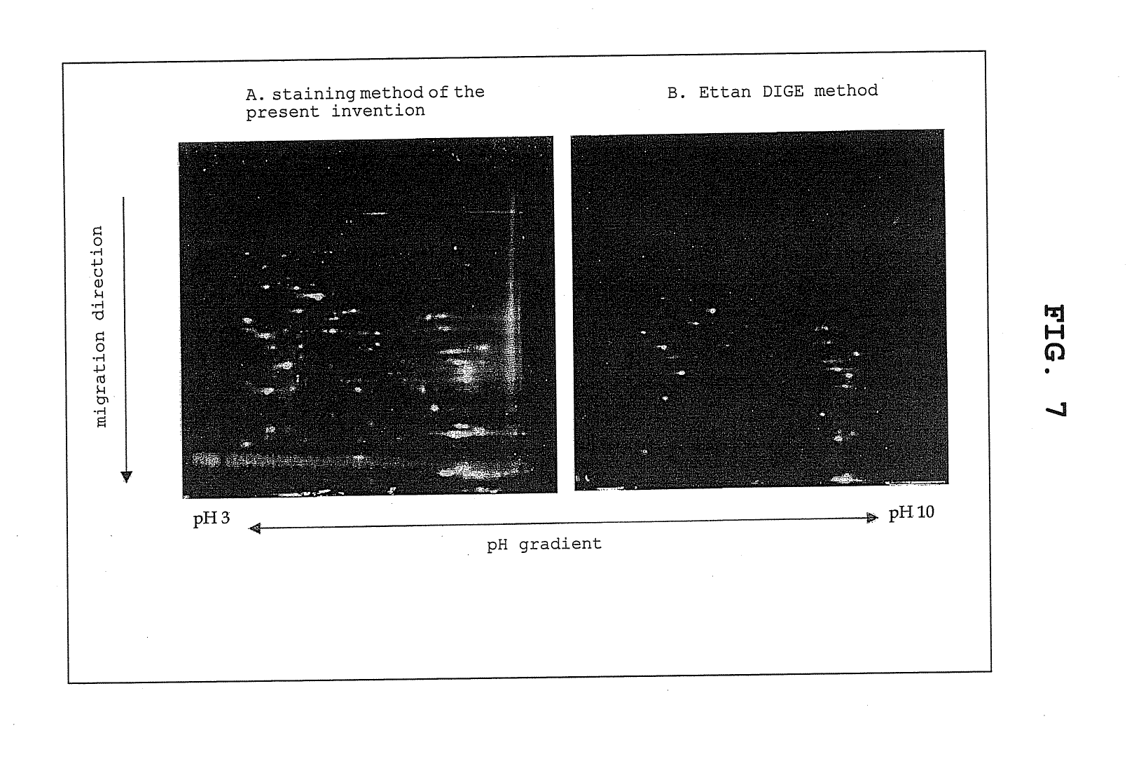

After the electrophoresis, the IPG gel strip was shaken for 15 min in an LDS equilibration buffer solution (1 mL, 4×LDS (manufactured by Invitrogen) 250 μL/H₂O 750 μL/2-mercaptoethanol 10 μL), and applied to the second dimension 4-12% gradient SDS-polyacrylamide electrophoresis gel, and electrophoresis was performed under the conditions of 200 V 40 min. After the electrophoresis, the fluorescence intensity at 680 nm was measured using a fluorescence image analyzer ProExpress (Perkin-Elmer) at an excitation wavelength 625 nm, based on which the migration image of the protein was observed. On the other hand, in the staining method of the present invention, two-dimensional electrophoresis was performed according to the aforementioned Examples and using the same weight of a protein extract. The migration image of the protein was observed using a fluorescence image analyzer and compared with the Ettan DIGE method. As a result, it was clarified that the migration image (FIG. 7A) obtained by the staining method of the present invention could be detected with higher sensitivity than the migration image (FIG. 7B) obtained by the Ettan DIGE method. In other words, it has been clarified that the staining method of the present invention permits staining by an operation in a short time as compared to the Ettan DIGE method, and detection with high sensitivity.

From the above-mentioned Examples, it has been clarified that the staining method of the present invention comprising two-dimensional electrophoresis, wherein a protein is brought into contact with a staining liquid between the first dimension and the second dimension, enables observation of a migration image of the protein separated by two-dimensional electrophoresis in a short time.

### INDUSTRIAL APPLICABILITY

According to the present invention, using a staining liquid containing a surfactant such as SDS and the like, a protein-containing sample can be stained in a short time, and can develop color with high sensitivity. In addition, since an excess staining agent migrates earlier than protein by electrophoresis, a washing operation is not necessary. Furthermore, by staining, after the first dimension electrophoresis, with a staining liquid containing a surfactant such as SDS and the like, the second dimension electrophoresis can be performed immediately. As a result, the number of steps can be reduced as compared to conventional protein separation methods, and the separation operation can be simplified. Hence, a method capable of staining and separating a protein conveniently and quickly by electrophoresis can be provided. In addition, a protein staining liquid and a protein staining kit useful for the protein staining and separation methods of the present invention can be provided.

This application is based on a patent application No. 2004-353395 filed in Japan, the contents of which are incorporated in full herein by this reference.

1. A method of separating a protein by gel electrophoresis, which comprises a staining step for bringing a protein-containing sample into contact with a staining liquid containing a staining agent, a surfactant and a buffer solution, and an electrophoresis step for subjecting the protein-containing sample after the staining step to gel electrophoresis.

2. The method of claim 1, wherein the surfactant is a dodecylsulfate alkali metal salt.

3. The method of claim 2, wherein the dodecylsulfate alkali metal salt is sodium dodecylsulfate or lithium dodecylsulfate.

4. The method of claim 2, wherein the concentration of the dodecylsulfate alkali metal salt in the staining liquid is 0.5-10%.

5. The method of claim 1, wherein the concentration of the buffer solution in the staining liquid is not more than 10 mM.

6. The method of claim 1, wherein the staining agent is a covalently-bonding staining agent.

7. The method of claim 6, wherein the covalently-bonding staining agent is an amino group-modifying staining agent.

8. The method of claim 7, wherein the amino group-modifying staining agent is a cyanine pigment.

9. The method of claim 1, wherein the gel electrophoresis is SDS-polyacrylamide gel electrophoresis.

10. The method of claim 1, which further comprises a step for subjecting the protein-containing sample to electrophoresis before the staining step, wherein the protein-containing sample in the staining step is the protein-containing sample after the electrophoresis.

11. The method of claim 10, wherein the electrophoresis is isoelectric focusing electrophoresis.

12. A method of staining a protein in gel electrophoresis, which comprises bringing a protein-containing sample into contact with a staining liquid containing a staining agent, a surfactant and a buffer solution before gel electrophoresis.

13. The method of claim 12, wherein the electrophoresis is two-dimensional electrophoresis comprising first dimension electrophoresis and second dimension electrophoresis, and the protein-containing sample after the completion of the first dimension electrophoresis is brought into contact with the staining liquid before the second dimension electrophoresis.

14. A protein-staining kit for gel electrophoresis, which comprises a buffer solution, a staining agent and a surfactant.

15. The protein-staining kit of claim 14, further comprising alcohol.

16. The protein-staining kit of claim 14, further comprising a written document containing an explanation of a method of separating a protein by gel electrophoresis, which comprises a staining step for bringing a protein-containing sample into contact with a staining liquid containing the buffer solution, the staining agent, and the surfactant, and an electrophoresis step for subjecting the protein-containing sample after the staining step to gel electrophoresis.

17. A protein-staining liquid for gel electrophoresis, comprising a staining agent, a surfactant and a buffer solution.

18. The protein-staining liquid of claim 17, further comprising alcohol.

19. The method of claim 3, wherein the concentration of the dodecylsulfate alkali metal salt in the staining liquid is 0.5-10%.

20. The protein-staining kit of claim 14, further comprising a written document containing an explanation of a method of staining a protein in gel electrophoresis, which comprises bringing a protein-containing sample into contact with a staining liquid containing the buffer solution, the staining agent, and the surfactant before gel electrophoresis.
